(19) United States
(12) Patent Application Publication
(10) Pub. No.: US 2014/0221906 A1
(43) Pub. Date: Aug. 7, 2014

(54) PATCH SYSTEM FOR THE TREATMENT OF WARTS AND METHOD FOR SAME

(71) Applicant: Jake W. Townsend, Manhattan Beach, CA (US)

(72) Inventor: Jake W. Townsend, Manhattan Beach, CA (US)

(21) Appl. No.: 13/914,183

(22) Filed: Jun. 10, 2013

Related U.S. Application Data
(60) Provisional application No. 61/761,522, filed on Feb. 6, 2013.

(51) Int. Cl. A61K 9/70; A61K 31/437; A61K 31/60; A61B 17/22; A61M 35/00

(52) U.S. Cl. CPC A61K 9/7084; A61K 9/7092; A61B 17/22; A61M 35/00; A61K 31/60; A61K 31/437
USPC 604/22; 604/307; 604/290; 514/161

(57) ABSTRACT

The present system and method relates in general to treating warts using a triple combination of therapies contained within a kit. The therapies are easy for a patient to self-apply, eliminating the need for constant physician supervision. The first therapy involves a patient applying a patch within the kit to a wart, which may be in the form of a transdermal patch. The patch may contain a first medicine, such as salicylic acid, and be worn for a period of twelve hours. Once removed, the second therapy involves using an ablative tool to gently slough away the treated wart, said ablative tool being contained within the kit. The third therapy involves applying a second patch, which may contain a second medicine, such as imiquimod, and also be worn for a period of twelve hours. This three step process may be repeated until the wart is fully treated.

### PRIORITY NOTICE

The present application claims priority under 35 U.S.C. §119(e) to U.S. Provisional Patent Application Ser. No. 61/761,522 filed on Feb. 6, 2013, the disclosure of which is incorporated herein by reference in its entirety.

### TECHNICAL FIELD OF THE INVENTION

The present system and method relate in general to the treatment of warts and more specifically to individuals who are able to self-treat warts via a combination of approved therapies with a prescription kit.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

Common and plantar warts affect up to ten percent of the population. They are caused by infection with the human papilloma virus (HPV) and can hamper a patient's quality of life by causing embarrassment, fear of negative appraisal by others, and frustration caused by persistence and/or recurrence. Moderate to extreme discomfort is reported in 51.7% of patients, and social or leisure activities are affected to a moderate to extreme degree in 38.8%. There is currently no cure for the HPV infection, and therapy does not affect transmissibility. Thus, current therapies aim at eliminating signs and symptoms, but no single treatment is fully effective in all patients. Different types of warts may need different treatments, and treatments often need to be combined.

Using a combination of therapies to treat patients with common and/or plantar warts may often be difficult, painful, and destructive to the skin tissue. © 2003 Stephen Tucker. Some treatments may include surgical curettage, cautery, caustic chemical ablation, and hyperthermic therapy. More specifically these treatments may include, but are not limited to, duct tape occlusion, exothermic patches, glutaraldehyde, formaldehyde, bacillus Calmette-Guŕin therapy, and levamisole. Other treatments may include cryotherapy, electrodessication, carbon dioxide laser ablation, paring, intralesional bleomycin, intralesional injection of interferon alfa, application of topical podophyllin, cantharidin, salicylic acid, and/or imiquimod. © 2003 Stephen Tucker. The physician may also employ other more simplified ablative therapies. The patient is often told to return to the physician's office once every one to three months for further ablative or cryotherapy.

Of those patients who have successful outcomes, many use combination therapies. Studies have shown that ablative therapies performed by a physician in combination with patient applied salicylic acid (concentrated at forty to sixty percent), imiquimod, and use of an ablative tool to be an effective treatment combination for common and plantar warts.

The physician may then suggest that the patient use over-the-counter wart remedies, including over-the-counter strength salicylic acid pads and/or duct tape occlusion, in combination with a topically applied prescription drug such as imiquimod. The patient then continues the at-home application of some combination of salicylic acid, imiquimod, ablation, and/or duct tape occlusion. The combined use of imiquimod, salicylic acid, and occlusion has shown to be an effective treatment for plantar warts that a patient can apply at home and thereby avoid other painful procedures.

Using these various prescriptions and methods, however, tends to be bothersome. The patient wastes time in applying each individual treatment. She also has to ensure that she is applying the correct treatment. She then has to make sure that the wart is adequately covered with an occlusive material. These issues may diminish the chances of effective treatment.

There is a need in the art for a system and method for home treatment of common and/or plantar warts that is efficient and effective and uses a combination of therapies using various medicines and an ablative tool. It is to these ends that the present system and method has been developed.

### BRIEF SUMMARY OF THE INVENTION

To minimize the limitations in the prior art, and to minimize other limitations that will be apparent upon reading and understanding the present specification, the present system and method describes a system for treating a wart with a kit comprising one or more first patches to be applied to said wart, and one or more second patches to be applied to said wart. Said kit further comprises an ablator, one or more holders, upon which said one or more first patches and said one or more second patches are placed, wherein said one or more holders are marked to correspond with a treatment cycle for said wart. Said kit further comprises one or more compartments for storing said one or more holders. Wherein said one or more compartments are marked to correspond with a treatment cycle for said wart.

Said one or more first patches comprise one or more first transdermal patches, wherein said one or more first transdermal patches comprise a first adhesive material for the purpose of securing said one or more first transdermal patches to said wart, and a first cavity to contain a first medicine, wherein said first medicine is released onto said wart after placement of said one or more first transdermal patches. Said one or more first transdermal patches further comprise a first membrane through which said first medicine may pass.

Said one or more second patches comprise one or more second transdermal patches, wherein said one or more second transdermal patches comprise a second adhesive material for the purpose of securing said one or more second transdermal patches to said wart, and a second cavity to contain a second medicine, wherein said second medicine is released onto said wart after placement of said one or more second transdermal patches. Said one or more second transdermal patches further comprises a second membrane through which said second medicine may pass.

The present system and method further describes a system for treating a wart with a multi-region, multi-layered transdermal patch.

It is an objective of the present system and method to make combination therapy for warts efficient and effective.

Is another objective of the present system and method to allow for a patient to self administer with ease combination therapies with an at home kit.

It is yet another objective of the present system and method to make combination therapy for warts as painless as possible.

These and other advantages and features of the present system and method are described herein with specificity so as to make the present system and method understandable to one of ordinary skill in the art.

### BRIEF DESCRIPTION OF THE SEVERAL VIEWS OF THE DRAWINGS

Elements in the FIGS. have not necessarily been drawn to scale in order to enhance their clarity and improve understanding of these various elements and embodiments of the apparatus and method. Furthermore, elements that are known to be common and well understood to those in the industry are not depicted in order to provide a clear view of the various embodiments of the system and method.

FIG. 1A is a three dimensional view of a patch kit.

FIG. 1B is a three dimensional view of an ablator.

FIG. 2A is a plan view of a patch kit, depicting compartments, holders, and patches.

FIG. 2B is a three dimensional view of an embodiment of a compartment within a patch kit containing two holders and four patches.

FIG. 3 is a plan view whereby holders gradually change color from a lighter to a darker shade.

FIG. 4 is a plan view of a holder and two patches.

FIG. 5A is a front elevation view of a patch depicting interior details.

FIG. 5B is a bottom view of a patch depicting a patch adhesive and a medicine.

FIG. 6 depicts a flowchart of the process one may undergo when applying a set of two patches containing different medicines.

FIG. 7A is a plan view a patch kit containing singular patches housing two different medicines.

FIG. 7B is a plan view of an aggrandized patch containing two different medicines.

FIG. 8A is a front elevation view of a patch containing two different medicines.

FIG. 8B is a bottom view of a patch depicting a patch adhesive, two medicines, and a barrier.

FIG. 9 depicts a flowchart of the process one may undergo when applying one patch containing two medicines.

FIG. 10A is a three dimensional view depicting a wart on a finger.

FIG. 10B is a three dimensional view depicting an application of a first patch containing medicine to a finger.

FIG. 11A is a three dimensional view depicting an ablator sloughing the skin off of a wart after an initial treatment with the patch in FIG. 10B.

FIG. 11B is a three dimensional view of a second patch applied to a wart containing a different medicine than the first patch in FIG. 10B.

FIG. 12A is a three dimensional view depicting the use of a patch containing two medicines.

FIG. 12B is a three dimensional view depicting an additional step when using a patch containing two medicines.

FIG. 13A is an illustration depicting the chemical structure of salicylic acid.

FIG. 13B is an illustration depicting the chemical structure of imiquimod.

### DETAILED DESCRIPTION OF THE INVENTION

In the following discussion that addresses a number of embodiments and applications of the present system and method, reference is made to the accompanying drawings that form a part thereof, where depictions are made, by way of illustration, of specific embodiments in which the system and method may be practiced. It is to be understood that other embodiments may be utilized and changes may be made without departing from the scope of the system and method.

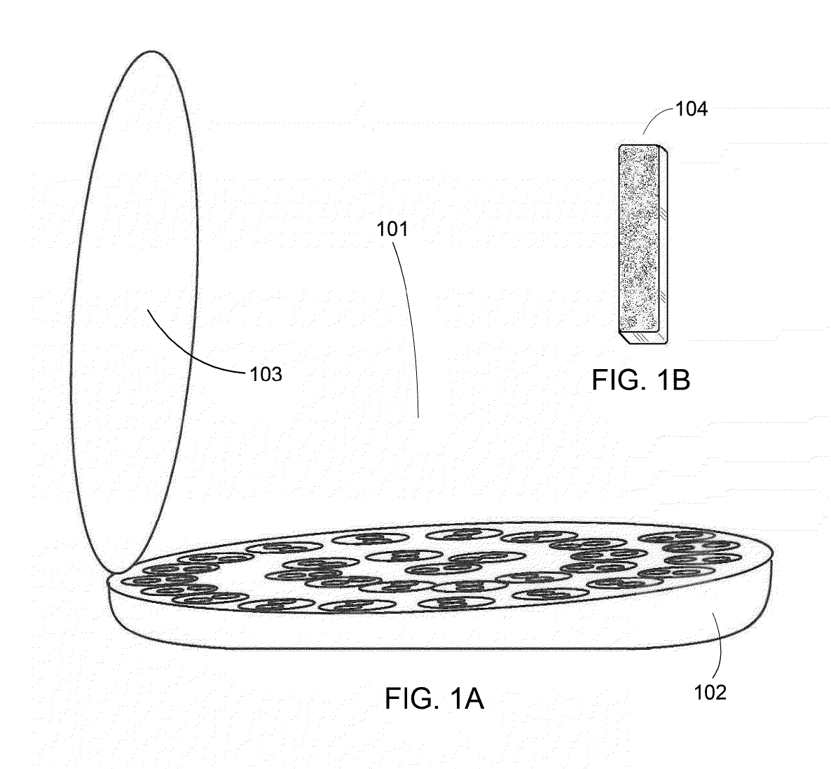

FIG. 1A is a three dimensional view of kit 101. FIG. 1B is a three dimensional view of ablator 104. Kit 101 may be comprised of container 102, and lid 103. Kit 101 including container 102 and lid 103 may further be comprised of plastic, or other natural or industrially produced substances. Container 102 and lid 103, however, need not be comprised of the same substances. For example, container 102 may be comprised of a more durable plastic while lid 103 may be lightweight plastic. Lid 103 may be connected to base 102 via several means. One may include using a hinge by which lid 103 is attached to base 102. Another may include lid 103 being in the form of a snap on lid, thereby eliminating the need for a hinge. Other means may be used to attach lid 103 to container 102.

Ablator 104 may be of any general structure such that it may accomplish its purpose of being able to file away undesirable skin. As such, ablator 104 may be in the form of an emery board, emery paper, nail file, pumice stone, sandpaper, or other similar forms.

In another embodiment, kit 101 and ablator 104 may be combined such that they are housed in the same container, such as a plastic bag or a box. For example, ablator 104 may be wrapped in cellophane like packaging, placed upon kit 101 and then kit 101, and ablator 104 may be wrapped again in cellophane like packaging and placed into the same box with one another. In another embodiment, ablator 104 may be attached to kit 101 such by a removable Glue Dot O. These methods, however, are not to limit the scope of how kit 101 and ablator 104 may be combined, as other methods of packaging may be employed that accomplish a similar purpose.

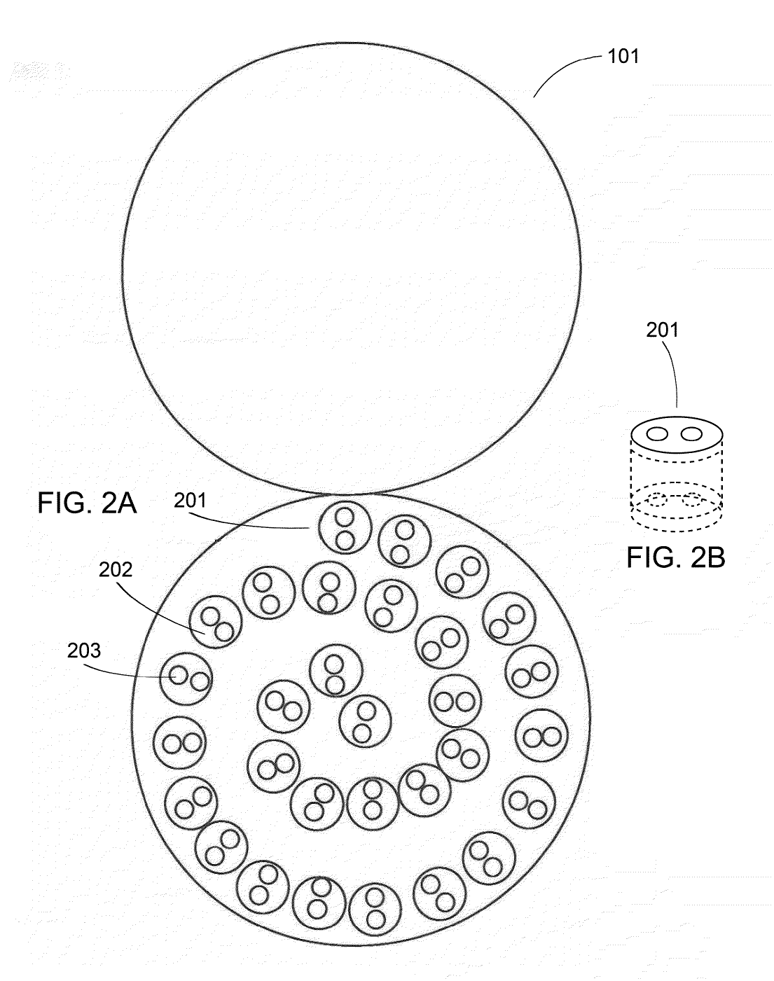

FIG. 2A is a plan view of kit 101 depicting compartment 201, holder 202, and patch 203. FIG. 2B is a three dimensional view of an embodiment of compartment 201 depicting two holders 202 and four patches 203. FIG. 2A further depicts the structure of kit 101. FIG. 2A depicts how compartments may be arranged in the form of a spiral or nautilus. Other arrangements are possible. For example, were kit 101 to be in the form of a square or rectangle, compartments 201 may be arranged in rows and columns. Other embodiments may utilize other means for compartments 201, such as where kit 101 is in the form of a box. Compartments 201 may be plastic baggies or individual containers capable of being removed from the box they may be housed in.

FIG. 2A also depicts thirty compartments 201. Each compartment 201 may correspond to one twenty-four hour period and hold one holder 202 and two patches 203, for example. As will be discussed further below, depending upon the nature of wart 1001 involved, more or less days may be needed to effectively treat wart 1001. As such, in the event that only one day is needed to treat wart 1001, kit 101 may only contain one compartment 201. Kit 101 may also contain more than thirty compartments 201 for similar reasons in the event that wart 1001 may take longer to treat. These alternative embodiments, however, are not to limit the scope of the present method and system.

FIG. 2B further depicts an embodiment of compartment 201 and a corresponding overall structure of kit 101. As opposed to compartment 201 being used to store a single patch 203, compartment 201 may be of such a depth that several patches 203 may be housed within a single compartment 201. For illustrative purposes, two patches 203 are depicted in FIG. 2B, however, it may be possible to store up to twelve patches 203 or more within a single compartment 201. As such, in the event that kit 101 were comprised of thirty compartments 201 that were of a depth such that twelve holders 202 could be housed within each compartment 201, this may make it possible for one kit 101 to effectively treat wart 1001 or a series of warts 1001 over the time span of nearly one year.

Apart from the structure, FIG. 2A further depicts the contents of kit 101. The contents of kit 101 may include a series of holders 202 and patches 203. Holder 202 may serve as a backing unit upon which patch 203 may rest and be held in place. Holder 202 may be comprised of such a material such that patch 203 is able to securely rest upon it. This material may be either synthetic or natural.

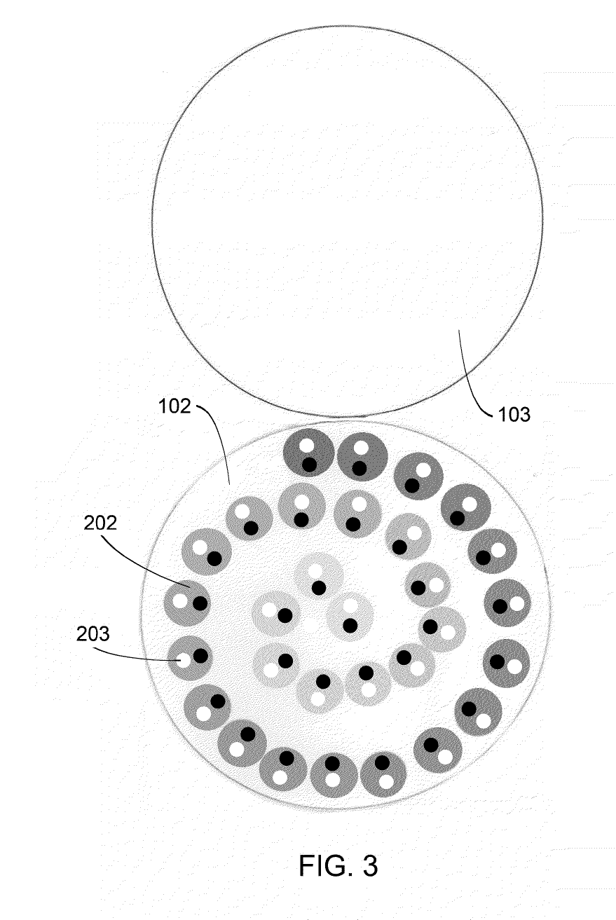

Holders 202 may also be distinguished from one another by several means. FIG. 3, for example, depicts a plan view whereby holders 202 gradually change color from a lighter to a darker a darker shade. Such a gradation in color may be beneficial to the user of patch 203, as it may be correlated with the progression of the treatment cycle, which may be supervised by a physician. In another embodiment, the actual coloration may vary such as from red, orange, yellow, green, blue, indigo and violet. Such gradation may also be used to correspond with a level of medicine 504 contained within patch 203. For example, in the beginning of a treatment cycle, a darker holder 202 may indicate that there is a higher level of medicine 504 in patch 203 than in a lighter colored holder 202. This may also be helpful when packaging kit 101 during the manufacturing process as it may help to avoid placing the wrong dosage of medicine 504 in the wrong compartment 201. In another embodiment, holders 202 may be of a uniform color and grade, however, compartments 201 may vary in color or gradation to serve similar purposes. In yet another embodiment, a marking such as a number or letter may be placed upon holder 202 or near compartment 201. Other identifying means may also be used.

Patch 203 may contain a certain level of medicine 504 for the treatment of wart 1001. Wart 1001 may be common or plantar. Wart 1001 may further be nongenital or nonperianal. Patch 203 may provide for an immediate or controlled release of medicine 504 from within patch 203 to the area of the skin that it is affixed to, as will be discussed further below. In various embodiments, medicine 504 may be comprised of cantharidin, glutaraldehyde, formaldehyde, formic acid, didanosine, stavudine and efavirenz or abacavir, bleomycin, retinoids, podophyllin, podophyllotoxin, oral zinc sulphate, dinitrochlorobenzene, diphencyorone, leukocytic interferon, candida antigen, fluorouracil, cimetidine, levamisole, topical bacillus Calumette-Guérin, 5-FU, interferon-α_{2b}, vidarabine, or trichophyton. Patch 203 may also contain a level of salicylic acid 1301 ranging in concentration from one to seventy percent, or a level of imiquimod 1302 ranging in concentration from one to four percent. Furthermore, patch 203 may be highly occlusive such as to ensure effective delivery of medicine 504 to wart 1001, and also to ensure effective overall treatment.

Various patches 203 within kit 101 may also contain various medicines 504. FIG. 2A, for example, depicts each holder 202 carrying two patches 203. One patch 203 may contain a level of salicylic acid 1301. The other patch 203 may contain a level of imiquimod 1302. Any medicine 504 discussed above, however, may be contained within patch 203. Furthermore, holder 202 may carry two patches 203 both containing salicylic acid 1301 while holder 202 to the immediate right or left may contain two patches 203 containing imiquimod 1302. At any rate, kit 101 may contain an equal amount of both salicylic acid 1301 and imiquimod 1302. This, in addition to ablator 104, may allow for a triple combination therapy to be implemented for the effective treatment of wart 1001. In a typical day, for example, a person may apply a “morning” patch 203 which may contain salicylic acid 1301, remove said morning patch 203, use ablator 104 to slough away the treated skin, and apply an “evening” patch 203, which may contain imiquimod 1302. The morning and evening patches 203 each may be worn for twelve hours respectively. This time interval may be lengthened or shortened in the event that varying levels and or types of medicines 504 are used with patch 203.

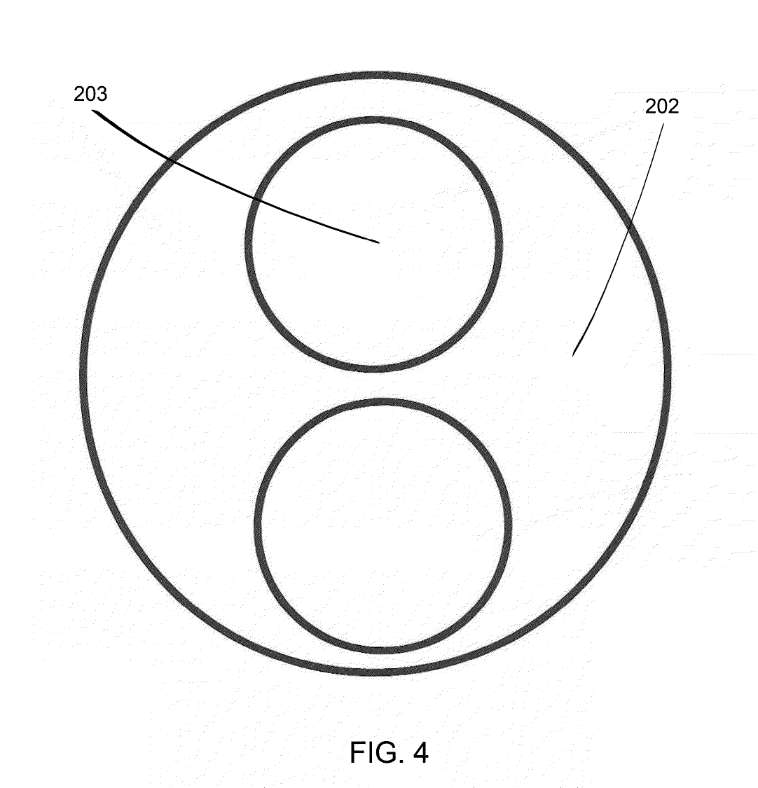

FIG. 4 is a plan view of holder 202 and two patches 203. This depiction, however, is not to limit the scope of the method and system. For example, in another embodiment holder 202 and patches 203 may be squared rather than rounded. Furthermore, more or less patches 203 may be placed upon holder 202, where the daily dose of medicine 504 requires more or less application or different types of treatments.

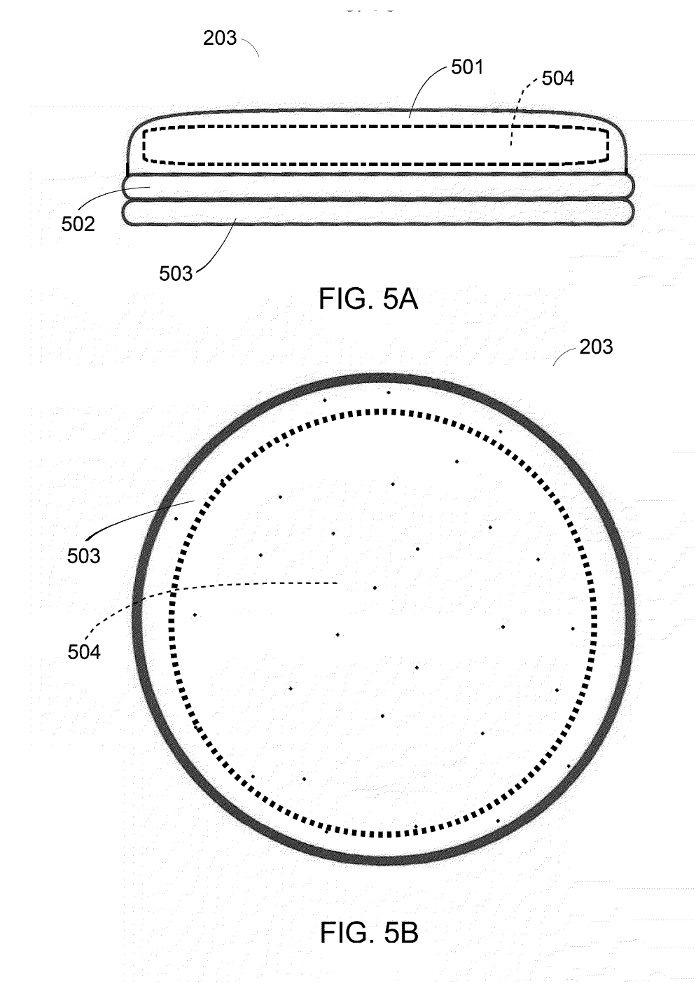

FIG. 5A is a front elevation view of patch 203 depicting interior details. This view depicts the components of which patch 203 may be comprised, including medicine cavity 501, membrane 502, patch adhesive 503, and medicine 504. Medicine cavity 501 may be comprised of a non porous material, such that medicine 504, which may be in the form of a liquid or gel is contained within patch 203. As depicted, medicine 504 may rest above membrane 502 and patch adhesive 503. Membrane 502 and patch adhesive 503 may be porous. A porous nature may allow for medicine 504 to seep through membrane 502 and patch adhesive 503 and be applied to wart 1001. The flow of medicine 504 may be activated, for example, by body heat. Patch 203, for example, may be of a transdermal nature.

FIG. 5B is a bottom view of patch 203 depicting patch adhesive 503, and medicine 504. As noted above, patch adhesive 503 may be porous in nature to allow medicine 504 to reach wart 1001.

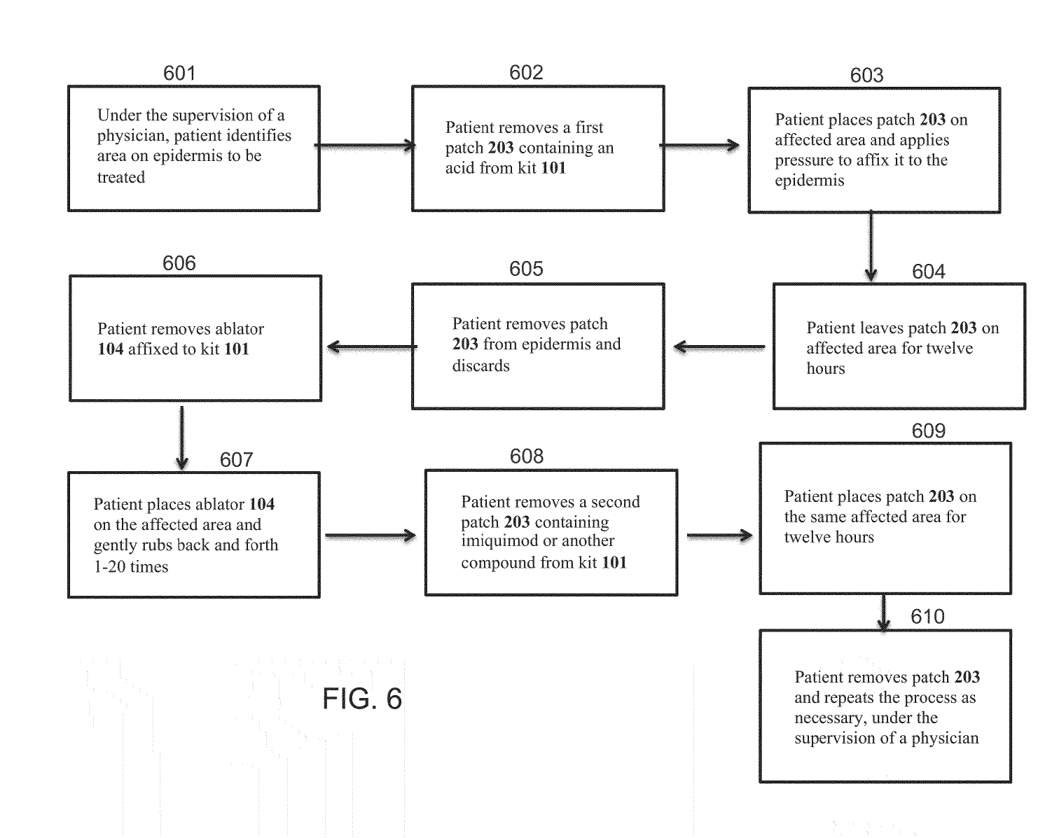

FIG. 6 depicts a flowchart of the process one may undergo when applying a set of two patches 203, as discussed in FIGS. 5A and B. Boxes 601 through 610 illustrate the steps of identifying where wart 1001 is located on the skin, and the process of the treatment cycle, which may be under the supervision of a physician until wart 1001 is eradicated. It may be noted that kit 101 may be available by prescription, or over-the-counter.

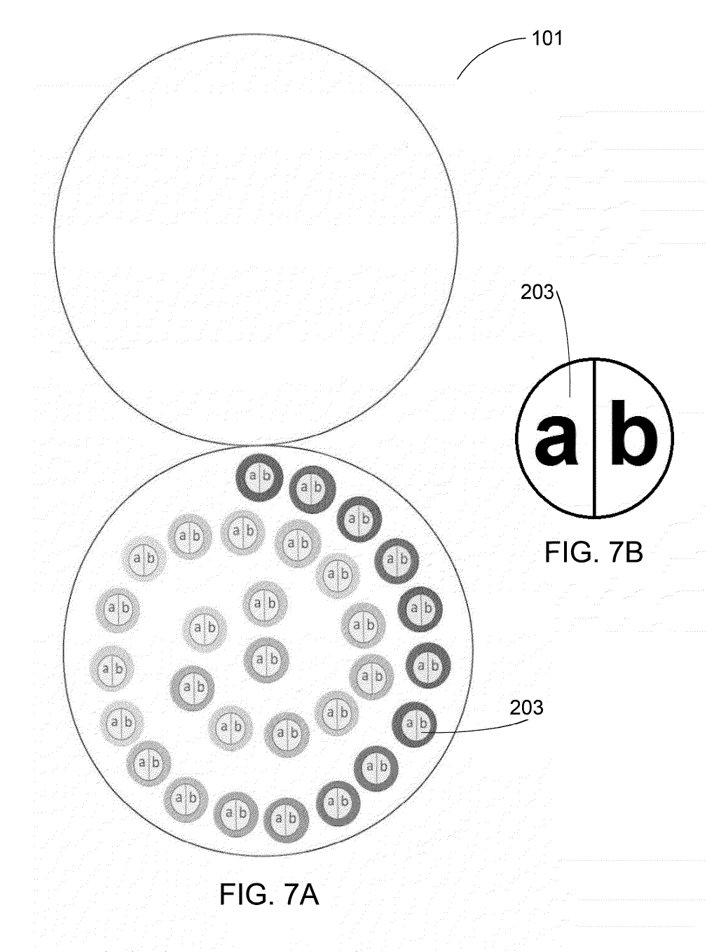

FIG. 7A depicts a plan view of an alternative embodiment, whereby kit 101 contains singular patches 203 that may house two different medicines 504. FIG. 7B is a plan view of an alternative embodiment of an aggrandized patch 203. Two different medicines 504 may be placed within patch 203, and indicated, for example, by an “a” and a “b” noted on the top of patch 203. Other markings may be utilized to indicate the different medicines 504. When prescribed, a doctor may inform a patient that the “a” represents salicylic acid 1301 while the “b” represents imiquimod 1302. This may also be indicated within the prescription itself. It may further be indicated on or around kit 101, such as on the box or within the packaging of kit 101.

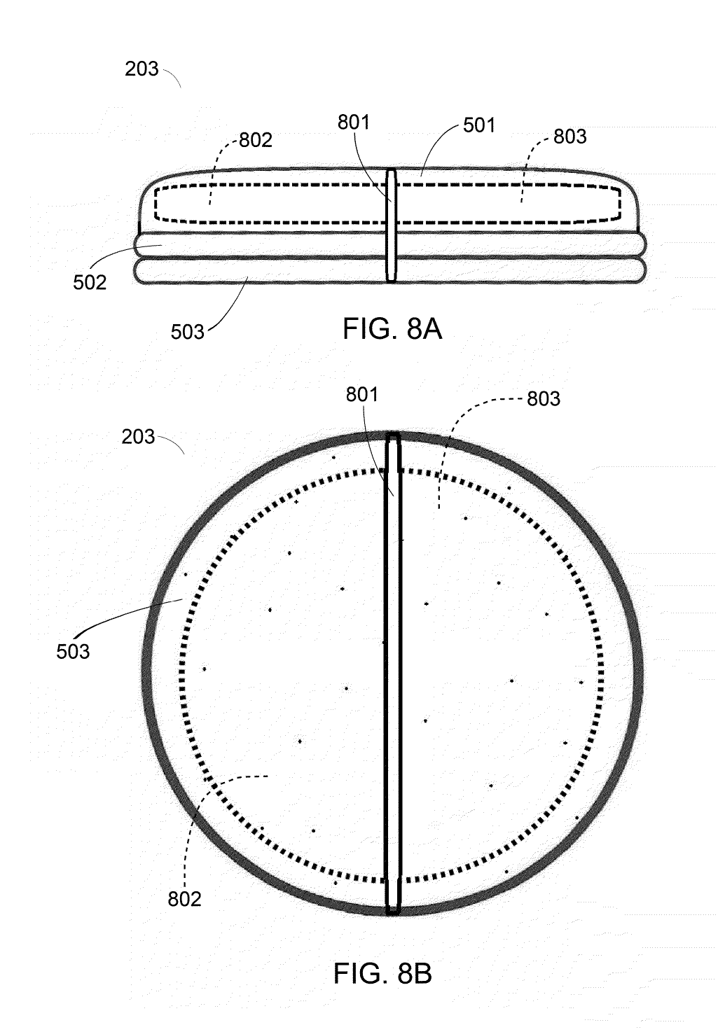

FIG. 8A is a front elevation view of the alternative embodiment of patch 203 described in FIG. 7B containing two medicines 504. An additional feature of this particular embodiment may include barrier 801. Barrier 801 may be a substance that keeps medicine “a” 802 separated from medicine “b” 803. Barrier 801 may further be an impermeable structure that spans the diameter of patch 203, as depicted in FIG. 8B, serving a similar purpose. FIG. 8B further depicts patch adhesive 503, medicine “a” 802, and medicine “b” 803.

Medicine “a” 802 and or medicine “b” 803 may be any of the medicines discussed thus far, including salicylic acid 1301 and imiquimod 1302. Medicine “a” 802 and or medicine “b” 803 may also be activated by body heat or some other mechanism such that the medicine is able to flow through membrane 502 and patch adhesive 503. Thus, the patches 203 depicted in FIGS. 8A and B may be transdermal in nature.

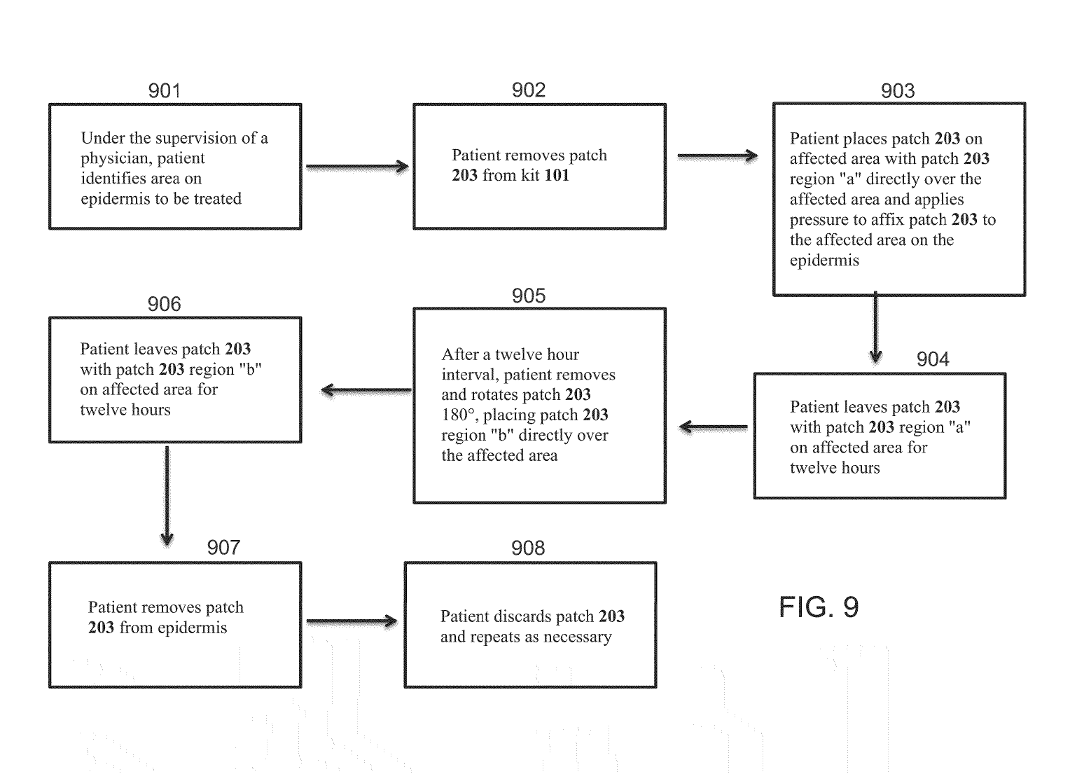

FIG. 9 depicts a flowchart of the process one may undergo when applying one patch 203 containing medicine “a” 802 and medicine “b” 803 as discussed in FIGS. 8A and B. Boxes 901 through 908 illustrate the steps of identifying where wart 1001 is located on the skin and the process of the treatment cycle, which may be under the supervision of a physician until wart 1001 is eradicated. A patient, however, may self administer treatments.

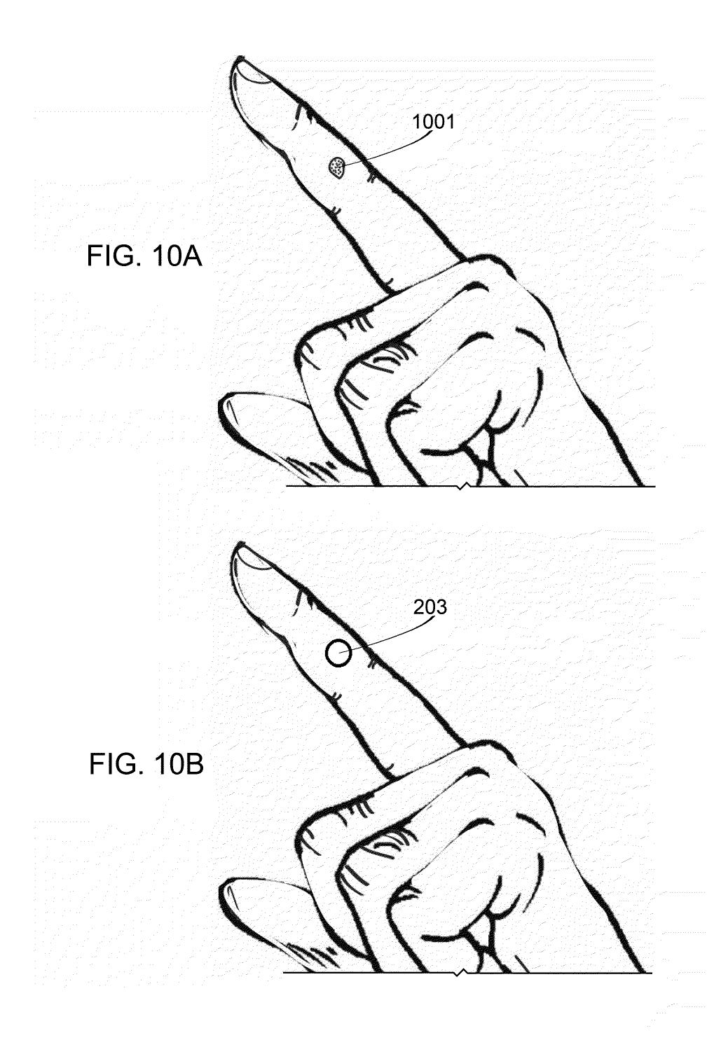

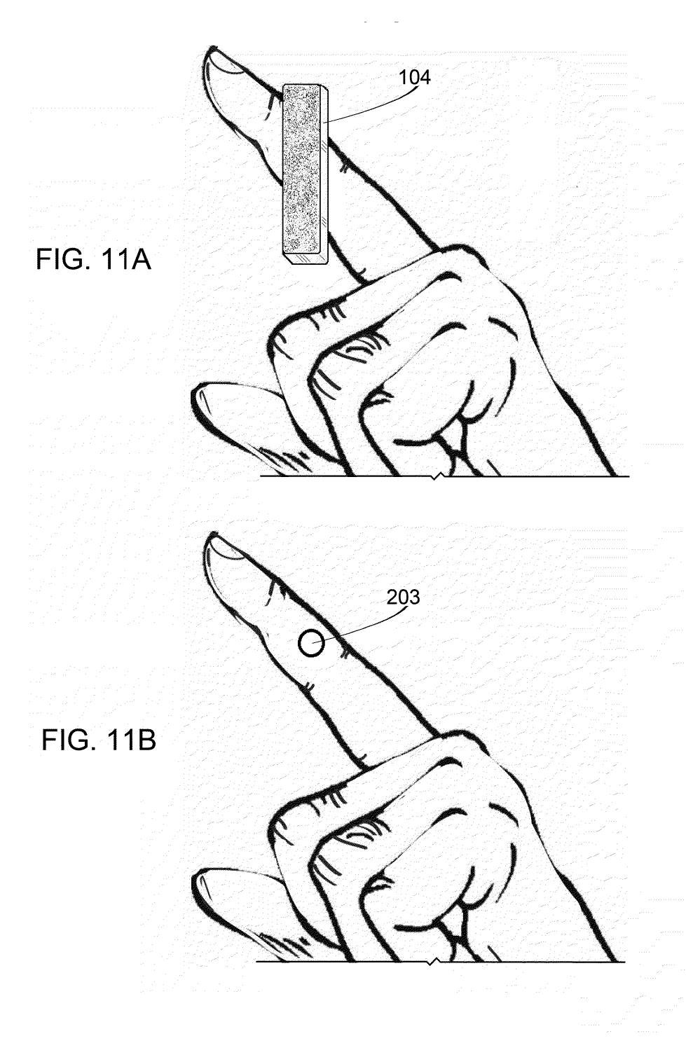

FIG. 10A is a three dimensional view depicting wart 1001 on a finger. FIG. 10B is a three dimensional view depicting an application of a first patch 203 containing medicine 504 to a finger. This may be considered the first therapy in this series of illustrations from FIG. 10A to FIG. 11B. This first application of patch 203 may contain, for example, salicylic acid 1301, and be worn for a period of twelve hours. It may also be noted that patch 203 may be of a small size, such that it is easily wearable. A small size may also be inconspicuous, allowing the wearer of patch 203 to avoid any social stigma associated with wart 1001.

FIG. 11A is a three dimensional view depicting ablator 104 sloughing the skin off of wart 1001 after an initial treatment with patch 203 in FIG. 10B. This may be considered the second therapy is this series of illustrations from FIG. 10A to FIG. 11B. As ablator 104 may be packaged together with kit 101, using it as such may add a level of convenience. Once sloughing is complete, a second patch 203 with a different medicine 504, such as imiquimod 1302, may be applied to wart 1001, as depicted by the three dimensional illustration in FIG. 11B. This second patch 203 may also be worn for a period of twelve hours. This may be considered the third therapy in this series of illustrations from FIG. 10A to FIG. 11B. After twelve hours, patch 203 may be removed, ablator 104 may be employed yet again, and another patch 203 containing salicylic acid 1301 may be applied to wart 1001. This process may continue for a period of one day, or may be repeated daily for up to one year or longer, until wart 1001 has been fully treated. This combination of therapies may be more likely to be an effective treatment for the eradication of the signs and symptoms of wart 1001.

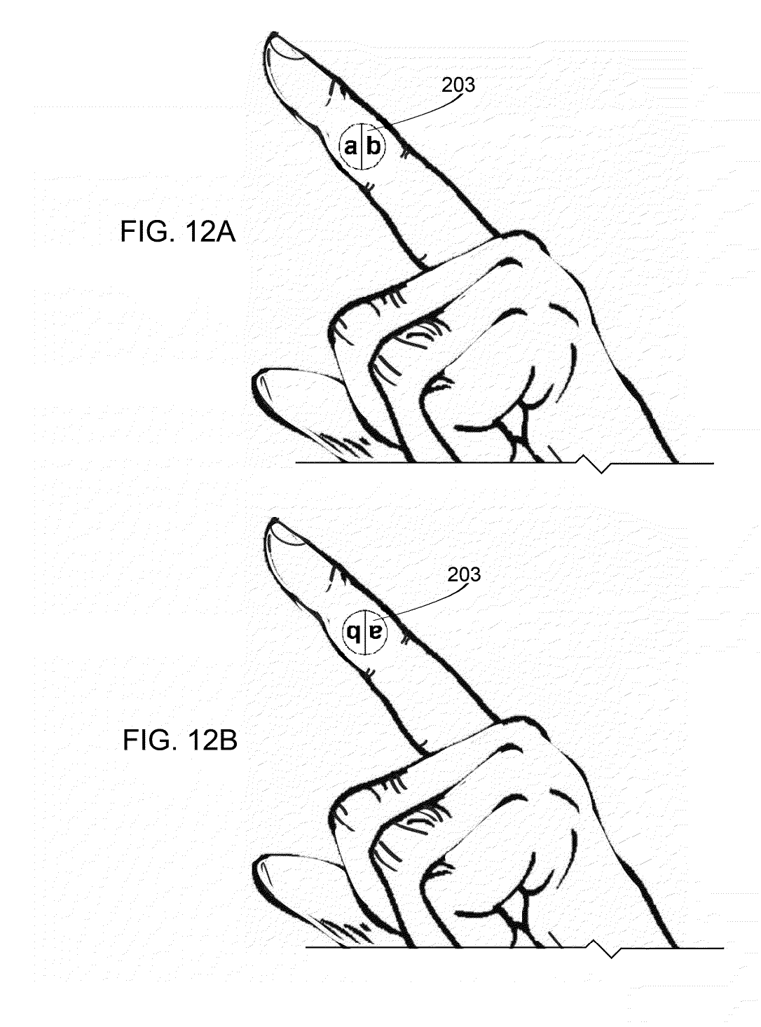

FIG. 12A is a three dimensional view depicting an alternative embodiment of the use of patch 203 containing two medicines 504. Patch 203 may contain both salicylic acid 1301 and imiquimod 1302, represented respectively by medicine “a” 802 and medicine “b” 803, separated by barrier 801. This may be considered a dual treatment as two medicines 504 are employed at the same time. After a period of twelve hours, patch 203 may be removed and wart 1001 may be sloughed with ablator 104 constituting the third therapeutic treatment. Patch 203 may then be reversed, as depicted in FIG. 12B such that medicine “a” 802 may treat where medicine “b” 803 was initially treating wart 1001 and medicine “b” may treat where medicine “a” was initially treating wart 1001. After a period of twelve hours, the process may be repeated with a fresh patch 203 containing a duality of medicines 504 until wart 1001 is fully treated.

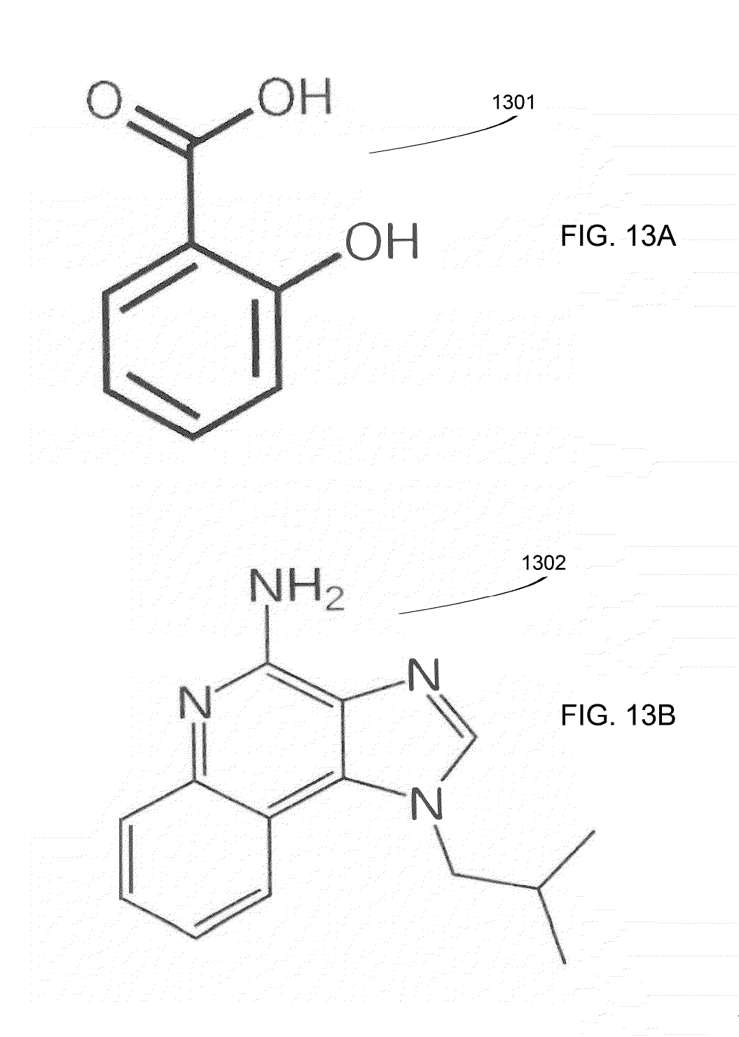

FIG. 13A is an illustration depicting the chemical structure of salicylic acid 1301. FIG. 13B is an illustration depicting the chemical structure of imiquimod 1302. Although these two medicines 504 may be effective in the treatment of wart 1001, other medicines 504 as discussed above with varying chemical structures may be equally effective.

A system and method for treating warts using various medicines has been described. The foregoing description of the various embodiments of the system and method has been presented for the purposes of illustration and disclosure. It is not intended to be exhaustive nor to limit the invention to the precise form disclosed. Many variations are possible in light of the above teaching. It will be understood that these variations in the system and method can be made by those skilled in the art without departing from the spirit of this system and method.

What is claimed is:

1. A system for treating a wart with a kit comprising:
one or more first patches to be applied to said wart; and
one or more second patches to be applied to said wart.

2. The system of claim 1, wherein said wart is a nongenital, nonperianal wart.

3. The system of claim 1, wherein said kit further comprises an ablator.

4. The system of claim 1, wherein said kit further comprises one or more holders, upon which said one or more first patches and said one or more second patches are placed.

5. The system of claim 4, wherein said one or more holders are marked to correspond with a treatment cycle for said wart.

6. The system of claim 5, wherein said kit further comprises one or more compartments for storing said one or more holders.

7. The system of claim 6, wherein said one or more compartments are marked to correspond with a treatment cycle for said wart.

8. The system of claim 1, wherein said one or more first patches comprise one or more first transdermal patches.

9. The system of claim 8, wherein said one or more first transdermal patches comprise:
a first adhesive material for the purpose of securing said one or more first transdermal patches to said wart; and
a first cavity to contain a first medicine, wherein said first medicine is released onto said wart after placement of said one or more first transdermal patches.

10. The system of claim 9, wherein said one or more first transdermal patches further comprise a first membrane through which said first medicine may pass.

11. The system of claim 10, wherein said first cavity contains salicylic acid.

12. The system of claim 11, wherein said one or more second patches comprise one or more second transdermal patches.

13. The system of claim 12, wherein said one or more second transdermal patches comprise
a second adhesive material for the purpose of securing said one or more second transdermal patches to said wart; and
a second cavity to contain a second medicine, wherein said second medicine is released onto said wart after placement of said one or more second transdermal patches.

14. The system of claim 13, wherein said one or more second transdermal patches further comprises a second membrane through which said second medicine may pass.

15. The system of claim 14, wherein said second cavity contains imiquimod.

16. A multi-region, multi-layered transdermal patch for treating a nongenital, nonperianal wart, comprising:
a top layer comprising:
a first region to contain a first medicine for the purpose of treating said wart; and
a second region to contain a second medicine for the purpose of treating said wart;
a middle layer having a membrane to contain said first and second medicine;
a bottom layer having an adhesive material for the purpose of attaching said multi-region, multi-layered transdermal patch to said wart;

17. The multi-region, multi-layered transdermal patch of claim 16, further comprising a barrier separating said first region from said second region, and wherein said first medicine is released onto said wart upon placement of said first region of said multi-region, multi-layered transdermal patch onto said wart, and said second medicine is released onto said wart after placement of said second region of said multi-region, multi-layered transdermal patch.

18. The patch of claim 17, wherein said first medicine cavity contains salicylic acid and said second medicine cavity contains imiquimod.

19. The patch of claim 18, wherein said first medicine cavity and said second medicine cavity are marked for identification.

20. A method of treating a wart with a cycle comprising the steps of:
placing a first transdermal patch having salicylic acid on a wart;
leaving said first transdermal patch on said wart for a period of time;
removing said first transdermal patch;
applying an ablative device to said wart for the purpose of removing a first treated layer of said wart;
placing a second transdermal patch having imiquimod on a wart;
leaving said second transdermal patch on said wart for a period of time;
removing said second transdermal patch;
applying said ablative device to said wart for the purpose of removing a second treated layer of said wart;
repeating said cycle if necessary until said wart is fully treated.
